(19) United States
(12) Patent Application Publication
(10) Pub. No.: US 2017/0249575 A1
(43) Pub. Date: Aug. 31, 2017

(54) MULTI CRITERIA DECISION ANALYSIS FOR MITIGATING OF CROWDING

(71) Applicant: HELSE STAVANGER HF, Stavanger (NO)

(72) Inventor: Olsen ØYSTEIN EVJEN, Kleppe (NO)

(73) Assignee: HELSE STAVANGER HF, Stavanger (NO)

(21) Appl. No.: 15/508,293

(22) PCT Filed: Sep. 2, 2015
(86) PCT No.: PCT/EP2015/070062

§ 371 Date: Mar. 2, 2017

Related U.S. Application Data
(60) Provisional application No. 62/044,511, filed on Sep. 2, 2014.

(51) Int. Cl. G06Q 10/06

(52) U.S. Cl. CPC G06Q 10/06313; G06Q 10/06375; G06Q 50/22

(57) ABSTRACT

It is disclosed a method and a computer program and user interface for handling and mitigating crowding at an institution. Based on customer flow indications and assessment of criticality of flow indicators, determinants responsible for crowding are determined. An aggregation of customer flow indicators is calculated. Interventions are calculated for mitigating the crowding. The aggregation, as well as customer flow indicators and proposed interventions are displayed to a user, for facilitating the decision process of determining which intervention to implement and when.

### TECHNICAL FIELD

This disclosure relates to multi criteria decision analysis for mitigating of crowding. In more particular, it relates to a method and a computer program for mitigating of crowding

### BACKGROUND

Hospital Emergency Department (ED) and hospital ward overcrowding has been subject to increased international focus over the past years. Key system challenges include increased number of patients utilizing the ED or hospital ward as the port of entry to hospital and specialized medical treatment, reduced availability of qualified staff and reduced availability of facilities and rooms. Hospital ED's and wards are increasingly becoming overcrowded resulting in reduced quality and patient safety, efficiency of services and trust, between service provider and the population in general, but also between health care providers.

Within this context there is a lack of decision-making tools providing the necessary analysis and support to handle and mitigate increased patient flow and health worker workload. Traditionally these decisions are left to clinical staff on an ad hoc and inconsistent basis. There is a void of tools displaying objective and useful information on an interactive technological platform relevant to the individual technical personnel, middle and top manager.

There is hence a need for an improved method to facilitate the decision-making process.

### SUMMARY

It is an object of embodiments of the invention to address at least some of the issues outlined above, and this object and others are achieved by a method for handling and mitigating crowding at an institution, and a computer program for handling and mitigating said crowding, according to the appended independent claims, and by the embodiments according to the dependent claims.

According to a first aspect, the disclosure provides a method for handling and mitigating crowding at an institution, wherein a flow of customer indicators are being observed, and wherein a number or resources are utilized. The method comprises obtaining real-time customer flow indicator input, and assessing current customer flow indicators and resource availability information. The method also comprises assessing a critical level for each customer flow indicator, and determining determinants responsible for crowding, wherein the determinants comprises customer flow indicators, based on the resource availability and the customer flow indicators together with the critical levels for each customer flow indicator. Further, the method also comprises calculating an aggregation of customer flow indicators of the determinants responsible for crowding, based on a multi criteria decision analysis (MCDA) methodology, and calculating interventions for mitigating of the crowding, based on the determined determinants responsible for the crowding and the calculated aggregation of customer flow indicators. Also, it comprises displaying most crucial determinants responsible for crowding, the calculated aggregation of customer flow indicators and the calculated interventions to a user, wherein each intervention is predicted to at least partly mitigate the crowding at the institution. In addition, the method comprises implementing an intervention according to received user decision input based on displaying most crucial determinants, calculated aggregation and calculated interventions, for mitigating the crowding.

According to a second aspect, this disclosure provides a computer program that comprises computer program code which, when run in a processor, causes a computer to obtain real-time customer flow indicator input, and assess current customer flow indicators and resource availability information. The program further causes the computer to assess a critical level for each customer flow indicator, and determine determinants responsible for crowding, wherein the determinants comprises customer flow indicators, based on the resource availability and the customer flow indicators together with the critical levels for each customer flow indicator. It also causes the computer to calculate an aggregation of customer flow indicators of the determinant responsible for crowding, based on a multi criteria decision analysis (MCDA) methodology, and calculate interventions for mitigating of the crowding, based on the determined determinants responsible for the crowding and the calculated aggregation of customer flow indicators. It also causes the computer to display most crucial determinants responsible for crowding, the calculated aggregation of customer flow indicators and the calculated interventions to a user, wherein each intervention is predicted to at least partly mitigate the crowding at the institution. In addition, it causes the computer to implement an intervention according to received user decision input based on displaying most crucial determinants, calculated aggregation and calculated interventions, for mitigating the crowding.

### BRIEF DESCRIPTION OF THE DRAWINGS

Embodiments will now be described in more detail, and with reference to the accompanying drawings, in which:

FIG. 1 presents a diagram over a typical flow of customers during a 24-hour period at an Emergency Department, on which customer flow expected or normal level of staffing and available resources is based;

FIG. 2 illustrates examples of actual and expected distributions of customer flow indicators describes an overcrowding situation in which levels of flow exceed planned availability of resources and flow infra-structure. The Figure also indicate a logical expression to forecast customer or patient flow using deviation/variance from normal increase (linearity of slope) or from normal acceleration (curve of slope) of customer flow indicators;

FIG. 3 illustrates key input, analysis and decision making processes in a continuous monitoring of and mitigation of crowding, according to some embodiments of the present invention;

FIG. 4 illustrates a flow-chart of a method for mitigating crowding, according to embodiments of the invention;

FIGS. 5 and 6 present examples of real-time and forecasted/predicted colour coded display, respectively, of level of key determinants/indicators, including dis-aggregated and MCDA aggregated levels. Information on pre-defined interventions/action points for each determinant is displayed, as well as contact details for key personnel being responsible for acting upon the information displayed through a user-friendly, computer generated interface of the present disclosure;

FIG. 7 presents a table of some examples of customer flow indicators and their corresponding critical levels, according to embodiments herein; and

FIG. 8 presents an example of staged intervention of the method/tool based on the contextual situation at the institution/department.

### DETAILED DESCRIPTION

This disclosure is based on a hospital Emergency Department (ED) and/or hospital ward settings where overcrowding has been subject to increased international focus over the past years.

This disclosure is also related to the development of analysing data on pre-defined criteria through a user-friendly interface for the implementation of generated action points to respond to and mitigate customer crowding. This disclosure relates to multi criteria decision analysis (MCDA) decision-making design and is preferably implemented in state of the art computer technology.

There is thus an urgent need to introduce a procedure combining a procedural, context relevant input solution and technical, computer, software and visual platforms based solution to improve the availability of information necessary for decision makers and clinical leadership within an ED, ward management and other customer related contexts to be prepared to handle and mitigate increased customer flow specified as number of customers, severity, such as workload, of each customer and barriers to flow.

In the following description, different embodiments of the invention will be described in more detail, with reference to accompanying drawings. For the purpose of explanation and not limitation, specific details are set forth, such as particular examples and techniques in order to provide a thorough understanding.

Embodiments of the present invention encompass a solution that is different from previous descriptions/applications of similar challenges. This solution does not seek to protect links in the process chart itself i.e. putting in personnel with specific functions to alleviate flow and bottleneck issues. While this is of interest, it is not the essence of this application.

Similarly, prior art simulation model generation solutions are overly general and do not involve the explicit process, analytical models nor the proposed visualization and application (app) solution as disclosed in this application. This application introduces, as an example, the qualitative and quantitative combination of information presented in the application to further improve the use of experience and include contextual information to secure relevance for the particular user. This application also sets out to provide a pragmatic, user friendly, evidence based and realistic set of indicators without including the all of the possible sets of data into the analytical models.

Prior art attempts to describe solutions have also shown to be overly complex and theoretical without relevance to the everyday setting of the technical expert, middle and top manager.

In addition prior art attempts to mitigate and handle crowding have focused on infrastructural and needs and demands for increased resource availability only, without acknowledging the importance of a proper situational analysis of the processes involved in utilizing infrastructure and resources effectively particularly related to handling and mitigating crowding.

Hospital ED's and wards are thus increasingly becoming overcrowded resulting in reduced quality and patient safety, efficiency of services and trust, between service provider and the population in general, but also between health care providers. Within this context there is a lack of decision-making tools providing the necessary analysis and support to handle and mitigate increased patient flow and health worker workload. Traditionally these decisions are left to clinical staff on an ad hoc and inconsistent basis. There is hence a void of tools presenting objective and useful information on an interactive technological platform relevant to the individual technical personnel, middle and top manager.

Experience from these situations has shown that these tools are preferably criteria based in which these criteria are transparent and relevant. It is also an advantage that the analysis and presentation are consistent and intuitive, based on easily available data.

This disclosure is also different from other systems, methods and apparatuses for predicting capacity of resources in an institution in that it provides more than the actual information of customer flow through a computer interface, but takes it one step further to also analyse the information according to the main expected bottlenecks to promote adequate, quick and implementable interventions based on a predefined analysis of the severity of the current overcrowding situation. Additionally, it extends directly to the end user through an application available at all times and through all media solutions, such as a mobile phone, for example, smart phone and tablets. These embodiments do therefore, in contrast to other solutions, present a description of a multi criteria decision and risk management analysis in which indicators are derived from a specific set of weighted, (or otherwise processed, data.

Finally this disclosure deviates from other systems in that it also embodies a solution for continued evaluation, re-specification of the customer flow indicators and set action point levels, and thus improvement of the method, computer program and the computer program product itself. According to some embodiments, steps to ensure continued relevance to the end user as the context of the application changes are comprised. These contextual changes may primarily be related to changes to the described levels of the customer flow indicators, i.e. number of customers, severity/workload and barriers to flow. Changes to the described levels of each indicator may primarily be due to altered access to existing resources.

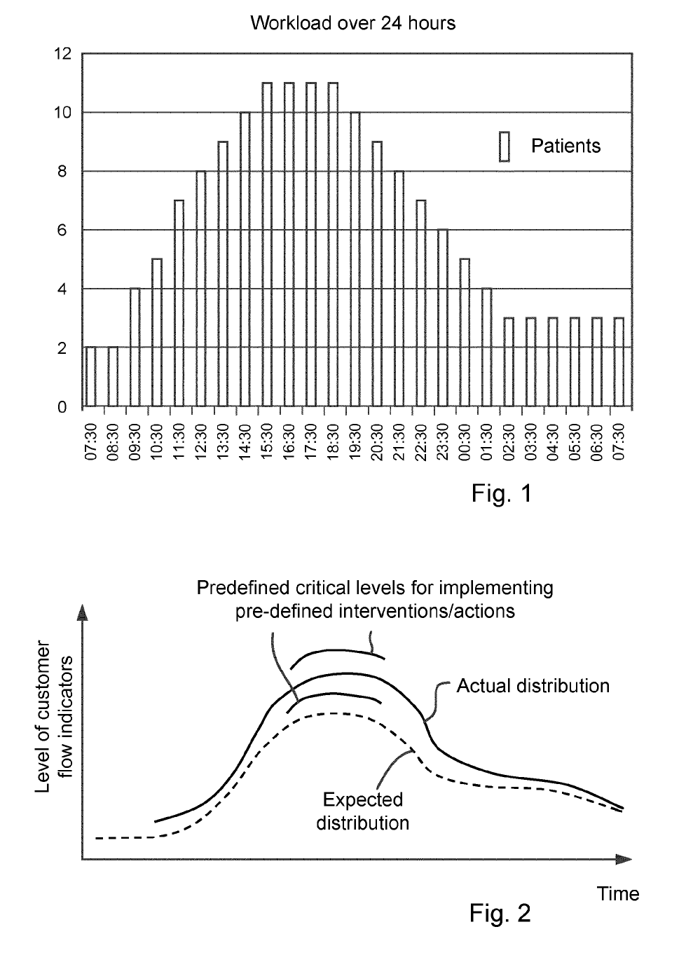

FIG. 1 presents a diagram over a typical flow of customers during a 24-hour period at an institution, such as, an Emergency Department, on which customer flow normal level of staffing and available resources is based.

The embodiments of the present invention describe a tool useful for middle managers as well as top managers to control and mitigate crowding at an institution, for example, in an Emergency Department, hospital ward or other customer relevant context, on a daily, continuous basis. The tool combines quantitative input from existing databases used in the institution to effectively create an overview of the most crucial determinants of crowding, to be analysed both qualitatively and quantitatively. To allow a qualitative analysis, the tool displays a disaggregated overview of the level of determinants both in real time and on a forecast basis. Based on this database input, technical experts, middle and top management and institutional leadership may determine where to implement effective interventions to regain control over the crowding situation in the institution. In addition the tool creates an overall aggregated score, based on a MCDA framework, for improved internal and external communication, e.g. to other wards, describing the situation in the institution for possible external assistance and wider decision making processes. Pre-defined interventions at each critical level are determined and displayed together with disaggregated determinants to facilitate receiving a quick, interactive response.

Embodiments of the present invention are based on a number of principles, including key stakeholders defining key information with a strong focus on the main institutional values such as trust, quality/customer safety and efficiency.

This key information is transformed through a legitimate, transparent and accountable analytical process to reflect scale up of emergency preparedness levels of crowding.

FIG. 2 illustrates examples of actual and expected distributions of level of customer flow indicators, for a crowded, or overcrowded, scenario. It is seen that the actual distribution is located at a level higher than the level of the expected distribution, being one relative measure of crowding, or overcrowding. There are also illustrated pre-defined critical levels for implementing pre-defined interventions/actions.

The analysis of information is characterized by pre-defined indicators, expressed through Monte Carlo, calculus expressed variance between expected and actual slope and curve of the crowding/overcrowding curve, as is illustrated in FIG. 2. The slope indicates the degree to which the indicators are increasing decreasing, and the curve of the slope the degree of acceleration of this increase or decrease. The indicators will be aggregated using MCDA methodology, potentially also based on weighting input variables against set parameters such as seriousness and likelihood of the identified risk.

This is done utilizing concepts of system based, strategic and operational risk analysis to identify cause and consequence, as well as barrier management. The information used for this purpose is narrowed down to pragmatically include information determining possible interventions for risk and crowding management. The information should specifically not be gathered through extensive, external processes, but from existing databases in the ED or hospital ward.

This will in turn provide real time and predictive information, both dis-aggregated and aggregated, to facilitate a qualitative and quantitative modality of selecting the most appropriate intervention to handle the particular, contextually defined overcrowding situation.

Over time the analytical process may be expressed through an iterative self-improvement system based on retrospective data over time to improve accuracy but also capture changes over time of an expressed “normal curve”. The typical normal patient flow curve, as of FIG. 1, will therefore be continuously adjusted to reflect a more accurate description of the variance between actual and normal values, both present and predicted.

The indicators will be defined according to the contextually available information in an institution database. Similarly critical levels determining the actions needed to be taken will be defined within the institution using the tool. This adaption process to the local context is critical to its usefulness as well as to the ownership and trust placed in the tool by the end users.

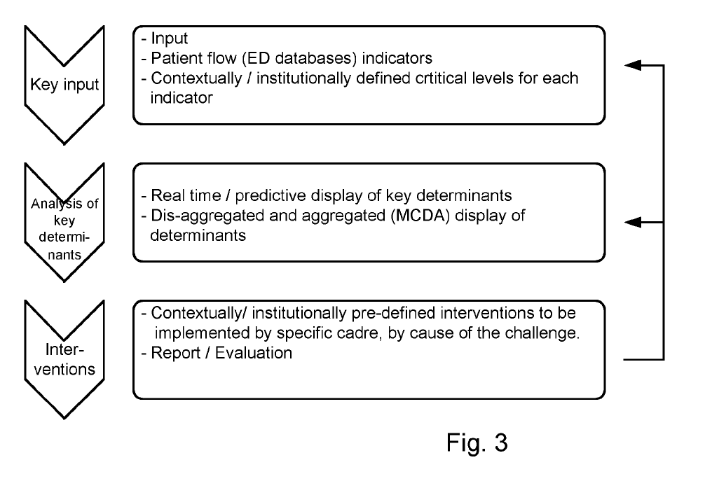

FIG. 3 illustrates key input, analysis of key determinants and interventions/decision making process in a continuous monitoring and mitigation of resource crowding, according to some embodiments of the present invention. The input involved may comprise user-defined description of the context specific indicators of customer flow, level of action points for each indicator, per-defined action points for each level and identification of and assignment of roles and responsibilities of each action point. The analysis involved may comprise collection of the relevant data for each of these indicators and subsequent computerized, mathematical process of providing a description of current and projected level for each of the customer flow indicators, such as level, severity, and barriers. The analysis may further be separated into a dis-aggregated and aggregated process to enable decision making at different managerial and technical levels. The interventions may also be presented through the visual, computerized interface of this disclosure to the end-user, to be a set of pre-defined interventions as developed within the institution and relevant to the end user. These interventions may be described and linked according to the level of each of the indicators, based on the analysis of key determinants of FIG. 3, involving presentation of pre-defined interventions and actions, based on the key input of FIG. 3.

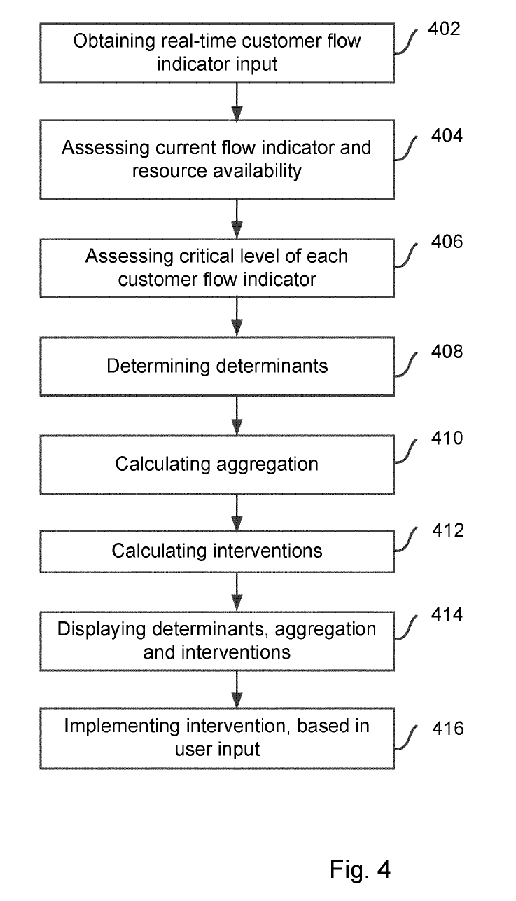

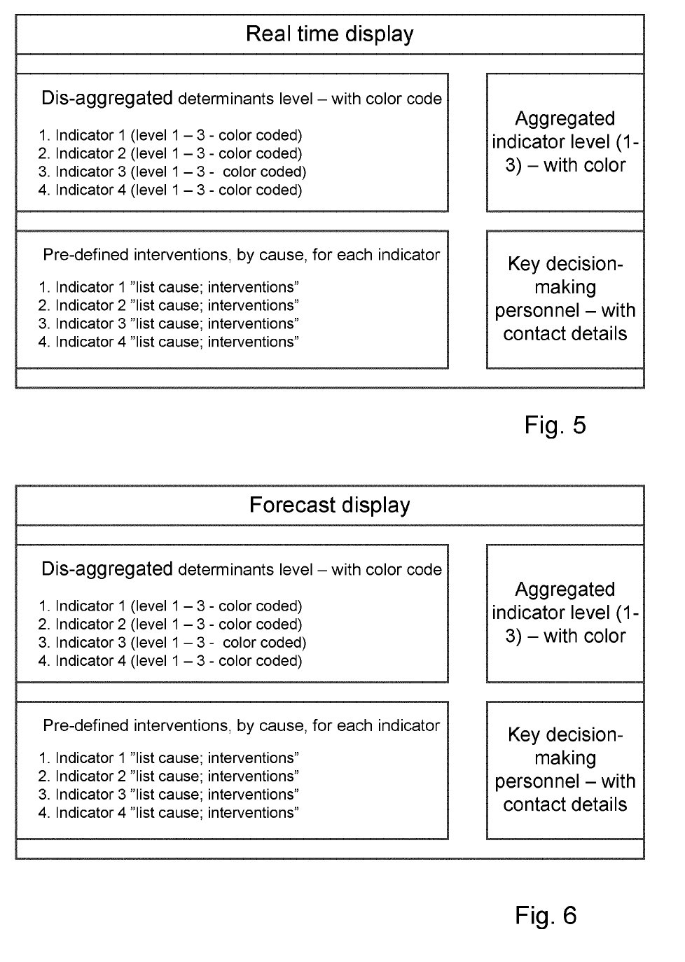

FIG. 4 presents a flow-chart of a method and a process for mitigating crowding. This disclosure thus provides a method and/or a process for mitigating crowding at an institution, wherein a flow of customer indicators are being observed, and wherein a number or resources are utilized. The method and/or comprises obtaining 402 real-time customer flow indicator input, and assessing 404 current customer flow indicators and resource availability information. The method and/or process also comprises assessing 406 a critical level for each customer flow indicator, and determining 408 determinants responsible for crowding, wherein the determinants comprises customer flow indicators, based on the resource availability and the customer flow indicators together with the critical levels for each customer flow indicator. Further, the method and also comprises calculating 410 an aggregation of customer flow indicators of the determinants responsible for crowding, based on a MCDA methodology, and calculating 412 interventions for mitigating of the crowding, based on the determined determinants responsible for the crowding and the calculated aggregation of customer flow indicators. Also, it comprises displaying 414 most crucial determinants responsible for crowding, the calculated aggregation of customer flow indicators and the calculated interventions to a user, wherein each intervention is predicted to at least partly mitigate the crowding at the institution. In addition, the method comprises implementing 416 an intervention according to received user decision input based on displaying most crucial determinants, calculated aggregation and calculated interventions, for mitigating the crowding.

Action 404 of assessing current customer flow indicators and resource availability information may comprise assessing the current customer flow indicators against the resource availability.

Action 404 of assessing resource availability information may comprise assessing the availability of doctors, for instance in waiting times; the availability of nurses, for example in waiting times; the availability of space, for instance in number of examination rooms; availability of equipment and/or availability of diagnostic services.

According to some embodiments some embodiments of the invention, the method as presented here may be a method for identifying, handling and mitigating crowding at an institution.

According to embodiments herein, action 414 also comprises displaying a level of the most crucial determinants responsible for crowding. This may be performed using colour-codes, where for instance a green coloured determinant or indicator, denotes a normal level, a yellow colour determinant or indicator denotes a level in which the determinant/indicator is to be considered, and a red coloured determinant or indicator denotes a level in which the determinant/indicator may have to be handled as soon as possible by performing one or more interventions.

According to embodiments herein, action 414 may comprise the level for each crucial determinant by numbers. Particular examples of such displayed determinants are the 1) #, i.e. number, of patient waiting more than one hour for a doctor, 2) # of patients waiting more than 3 hours in the department in question, 3) # of patients in the department, and 4) # of patients in triage severity categories, for instance red and orange.

According to some further embodiments of this invention, the method for handling and mitigating crowding at an institution comprises identifying indicators of the customer flow with the objective of describing level of flow, such as number of customers, severity of resource utilization, such as workload of each customer, and barriers to flow. Mechanisms for which these indicators will be pre-defined for each user in order to secure relevance and user applicability may also be provided. This method may further specify obtaining real-time said indicator input, and assessing level, severity and barrier indicators against resource availability information. The method may also comprise assessing a critical level for each level, severity and barrier indicator, and determining specific determinants responsible for crowding at the given time, wherein the determinants may comprise level, severity and barrier indicators. The critical levels are pre-defined, for instance by the user, to provide action points based on an assessment of resource availability. Further, the method also comprises calculating an aggregation of level, severity and barrier indicators of the determinants responsible for crowding, based on a MCDA methodology, and calculating the interventions likely to be most relevant for handling and mitigating the crowding, based on the determined determinants responsible for the crowding and the calculated aggregation of customer flow indicators.

The flow-chart as presented in FIG. 4 equally well describes steps of a process for mitigating crowding, for which reason the present disclosure also comprises a process.

Figure schematically presents examples of real-time colour coded display of level of key determinants/indicators, including dis-aggregated and MCDA aggregated levels. Information on pre-defined interventions/action points for each determinant is displayed, as well as contact details for key personnel.

FIG. 6 schematically presents a similar display of examples of forecasted/predicted colour coded display of level of key determinants/indicators, including dis-aggregated and MCDA aggregated levels. By analysing a distribution of customer flow indicator in terms of deviation/variance from a linear increase/decrease with respect to time, being the, so called, first time derivative, and from a curved increase/decrease with respect to time, being the so called second time derivative, a projected level of customer flow indicators for 1-3 hours may be calculated. Based on these projected levels, forecast or predicted determinants/indicators, including dis-aggregated and MCDA aggregated levels may be determined, as schematically illustrated in FIG. 6.

According to some embodiments, important determinants influencing the crowding indicators are further introduced into the analytical framework of the MCDA tool. As an example from an Emergency Department context these include number of patients, triage colour scheme, patient contamination status and supplementary tests, wherein triage refers to a priority setting tool. The colour scheme used to triage patients and describe their need for healthcare assistance may for instance have four colour codes according to their severity: red, orange, yellow and green.

The herein proposed action levels of each indicator have been configured within the context of the institution and may be implemented in 4 phases. In the case the institution is an Emergency Department, the first phase may define the number of patients that can be handled by healthcare personnel within the constraints of time, severity of the patient and availability of space. This phase may also include an institutional weighting process in which number of patients, triage colour code, contamination status and supplementary tests are each weighted to add to the level of expected crowding over time, for instance 3-6 hours. One example of severity of patients or customers is level of contamination status and thus generates workload, which in turn negatively impacts crowding, as these resources occupied for these purposes cannot be effectively used to handle other patients waiting to be served.

It is noted that individual levels with corresponding interventions are configured for each indicator at each institution implementing the tool. The tool may include a start-up configuration module including a training manual and video tutorial. This configuration module will be modifiable by the institution as the institutional context evolves over time.

Both the suspected cause of crowding and suggested intervention to handle and mitigate the situation will hence be displayed together with the colour code (level) of the indicator.

A brief but widely inclusive process has been undertaken during the development phase of the tool/method in order to arrive at indicators and critical levels needed to execute a verification study of the tool. The critical levels were determined based on a qualitative process, using the input of experienced personnel and simulation exercises, in which availability of personnel and space were the most critical factors identified at each level.

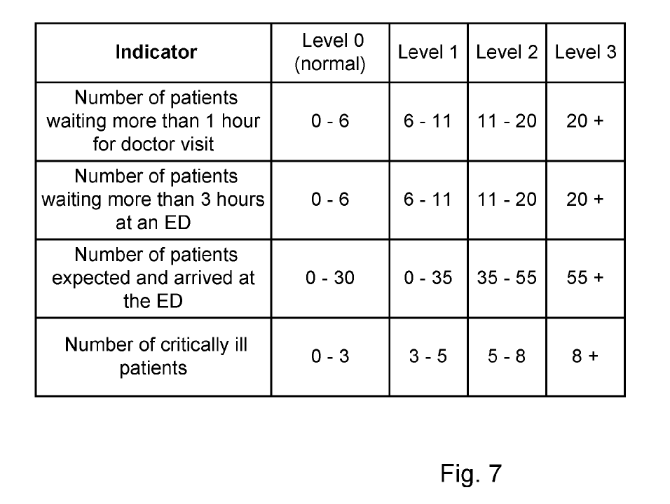

FIG. 7 presents a table of example indicators and corresponding critical levels for action, according to some embodiments of the present invention. The indicators for which the levels are presented comprise the number of patient waiting more than one hour for a doctor, the number of patients waiting more than 3 hours in the department in question, the number of patients in the department, and the number of patients in triage severity categories, for instance red and orange.

For this particular example it is noted that the South African triage system (SATS), as a basis for the indicators of FIG. 7, is a validated tool for determining patient situation and need for medical attention. This disclosure is flexible to any such determining tool within a customer related context.

The most common causes of crowding were identified. These causes further define the interventions needed to be implemented at each level, and were classified as follows:

1. Availability of doctors

2. Availability of nurses and auxiliary personnel

3. Availability of rooms and area or space

Several examples of indicators of varying levels, severity and barriers to flow may be thus envisaged, of which the ones in FIG. 7 are only a few. Together with potential shortage of specific qualified personnel such as anaesthetists, surgeons, physical resources, etc. main determinants of crowding may be determined.

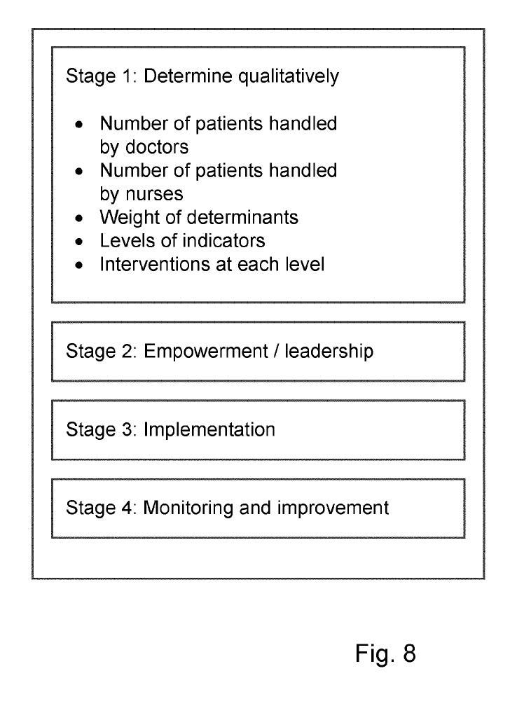

FIG. 8 presents a summary of the stages within the example presented above, summarizing staged intervention of the method as herein disclosed, to be relevant to a contextual situation at an institution/department.

Stage one may include the determination of the capability of doctors, nurses and infrastructure, for instance space and equipment, to handle patients, specifically identified as the number of patients. Weight of determinants relevant to crowding, such as contamination status, severity, number of tests, may then be applied to this assessment. The assessment may then proceed to provide a set of indicators relevant to describe the slope and curve of a crowding situation, as exemplified by the actual and expected distributions of FIG. 2. The staged intervention may further proceed to assign specific levels to each of these indicators, as was illustrated in FIG. 7, and finally present interventions relevant to each of these levels to handle and mitigate crowding.

The second stage embodied in this disclosure may comprise specific steps to ensure a wide ownership, empowerment and leadership dedication to the use of this disclosure, to secure its proper implementation and effect.

The third stage may involve description of implementation of preferable steps to secure successful implementation within the institution, involving evidence based implementation theory into this process.

The fourth stage embedded in this disclosure may comprise a monitoring and improvement mechanism to secure continued and sustained relevance and effect of embodiments of this invention within the institution.

The present disclosure also provides a computer program that comprises computer program code which, when run in a processor, causes a computer to obtain real-time customer flow indicator input, and assess current customer flow indicators and resource availability information. The program further causes the computer to assess a critical level for each customer flow indicator, and determine determinants responsible for crowding, wherein the determinants comprises customer flow indicators, based on the resource availability and the customer flow indicators together with the critical levels for each customer flow indicator. It also causes the computer to calculate an aggregation of customer flow indicators of the determinants responsible for crowding, based on a multi criteria decision analysis, MCDA, methodology, and calculate interventions for mitigating of the crowding, based on the determined determinants responsible for the crowding and the calculated aggregation of customer flow indicators. It also causes the computer to display most crucial determinants responsible for crowding, the calculated aggregation of customer flow indicators and the calculated interventions to a user, wherein each intervention is predicted to at least partly mitigate the crowding at the institution. In addition, it causes the computer to implement an intervention according to received user decision input based on displaying most crucial determinants, calculated aggregation and calculated interventions, for mitigating the crowding.

The program may further cause the computer to assess a critical level for each customer flow such as level indicator, severity indicator and barrier indicator.

The computer program may also cause the computer to display pre-defined interventions at each critical customer flow indicator level, according to received user decision input based on displaying most crucial determinants, calculated aggregation and calculated interventions, for mitigating the crowding.

The computer program may also cause the computer to display pre-defined interventions at each critical customer flow indicator level on an interactive user interface to immediately capture and reanalyse the effect on the customer flow indicators of decisions made by the end user.

It should be noted that the method and/or process, as described herein, may advantageously be combined with the computer program for mitigating of crowding.

The embodiments of present invention have the following advantages:

The applicability of a simple-to-use tool defining the critical level of crowding at an institution is clearly an advantage. Levels of severity are correlated with the most common causes of crowding, with associated interventions to handle and mitigate the crowding situation and regain control.

In addition, a further advantage is the process of developing critical values as well as the interventions, as these are contextually defined within the institution adapting the tool.

This ensures ownership, trust and relevance of the tool, ultimately increasing the usefulness of the tool in terms of securing quality, security and efficiency of services to the customers.

Embodiments of the present invention may accordingly provide a:

1. description of process involved in identifying key information;

2. description of key indicators used to assess level of crowding;

3. description of use and analysis of key information; and

4. description of display of information for decision making.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts may be performed as a single step and/or phase. Also, certain steps and/or phases may be broken into additional sub-components to be performed separately. In some instances, the order of the steps and/or phases may be rearranged and certain steps and/or phases may be omitted entirely. Also, the methods described herein are to be understood to be open-ended, such that additional steps and/or phases to those shown and described herein may also be performed.

Some aspects of the systems and methods described herein may advantageously be implemented using, for example, computer software, hardware, firmware, or any combination of computer software, hardware, and firmware. Computer software may comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that may be implemented using software to be executed on a general purpose computer may also be implemented using a different combination of hardware, software, or firmware. For example, such a module may be implemented completely in hardware using a combination of integrated circuits. Alternatively or additionally, such a feature or function may be implemented completely or partially using specialized computers designed to perform the particular functions described herein rather than by general purpose computers.

Multiple distributed computing devices may be substituted for any one computing device described herein. In such distributed embodiments, the functions of the one computing device are distributed (e.g., over a network) such that some functions are performed on each of the distributed computing devices. Some embodiments may be described with reference to equations, algorithms, and/or flowchart illustrations. These methods may be implemented using computer program instructions executable on one or more computers. These methods may also be implemented as computer program products either separately, or as a component of an apparatus or system. In this regard, each equation, algorithm, block, or step of a flowchart, and combinations thereof, may be implemented by hardware, firmware, and/or software including one or more computer program instructions embodied in computer-readable program code logic. As will be appreciated, any such computer program instructions may be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, may be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that may direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor, or multiple processors, that executes program instructions or modules stored in a memory or other non-transitory computer-readable storage medium or device. The various functions disclosed herein may be embodied in such program instructions, although some or all of the disclosed functions may alternatively be implemented in application-specific integrated circuits (ASICs) or filed-programmable gate arrays (FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of this disclosure may be applied to other methods and systems, and are not limited to the methods and systems described above, and elements and acts of the various embodiments described above may be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

### ABBREVIATIONS

ASIC Application specific integrated circuit

ED Emergency department

FPGA Field-programmable gate array

MCDA Multi criteria decision analysis

SATS South African triage system

1. A method for resource utilization mitigating crowding at an institution, wherein time-series development of customer flow indicators are being observed, and wherein a number or resources are utilized, the method comprising:
obtaining real-time customer flow indicator input;
assessing current customer flow indicators and resource availability information, using special purpose hardware and computer-readable program code logic means;
assessing a critical level for each customer flow indicator;
determining determinants responsible for crowding, wherein the determinants comprises customer flow indicators, based on the resource availability and the customer flow indicators together with the critical levels for each customer flow indicator;
calculating an aggregation of customer flow indicators of the determinants responsible for crowding, based on a multi criteria decision analysis, MCDA, methodology;
calculating interventions for mitigating of the crowding, based on the determined determinants responsible for the crowding and the calculated aggregation of customer flow indicators;
displaying most crucial determinants responsible for crowding, the calculated aggregation of customer flow indicators and the calculated interventions to a user, wherein each intervention is predicted to at least partly mitigate the crowding at the institution; and
implementing an intervention according to received user decision input based on displaying most crucial determinants, calculated aggregation and calculated interventions, for mitigating the crowding.

2. The method for mitigating crowding according to claim 1, wherein the determining of determinants responsible for crowding comprises a Monte Carlo analysis of a variance between an expected and actual slope of customer flow as a function of time, and on a variance between an expected and actual curve of customer flow as a function of time.

3. The method for mitigating crowding according to claim 1, wherein the determining of determinants comprises determining real-time determinants responsible for the crowding, and wherein the calculating comprises calculating real-time interventions for mitigating of the crowding.

4. The method for mitigating crowding according to claim 1, wherein the determining further comprises determining forecast determinants responsible for the crowding, and wherein the calculating comprises calculating forecast interventions for mitigating of the crowding.

5. A computer program comprising computer program code which, when run in a processor, causes a computer to:
obtain real-time customer flow indicator input;
assess current customer flow indicators and resource availability information;
assess a critical level for each customer flow indicator;
determine determinants responsible for crowding, wherein the determinants comprises customer flow indicators, based on the resource availability and the customer flow indicators together with the critical levels for each customer flow indicator;
calculate an aggregation of customer flow indicators of the determinant responsible for crowding, based on a multi criteria decision analysis, MCDA, methodology;
calculate interventions for mitigating of the crowding, based on the determined determinants responsible for the crowding and the calculated aggregation of customer flow indicators;
display most crucial determinants responsible for crowding, the calculated aggregation of customer flow indicators and the calculated interventions to a user, wherein each intervention is predicted to at least partly mitigate the crowding at the institution by resource utilization; and
implement an intervention according to received user decision input based on displaying most crucial determinants, calculated aggregation and calculated interventions, for mitigating the crowding.

6. A computer program product comprising a computer program according to claim 5 and computer readable means on which the computer program is stored.

7. An institution having software and hardware for improving resource utilization, wherein said software and hardware are adapted to perform the steps of:
obtaining real-time customer flow indicator input;
assessing current customer flow indicators and resource availability information;
assessing a critical level for each customer flow indicator;
determining determinants responsible for crowding, wherein the determinants comprises customer flow indicators, based on the resource availability and the customer flow indicators together with the critical levels for each customer flow indicator;
calculating an aggregation of customer flow indicators of the determinants responsible for crowding, based on a multi criteria decision analysis, MCDA, methodology;
calculating interventions for mitigating of the crowding, based on the determined determinants responsible for the crowding and the calculated aggregation of customer flow indicators;
displaying most crucial determinants responsible for crowding, the calculated aggregation of customer flow indicators and the calculated interventions to a user, wherein each intervention is predicted to at least partly mitigate the crowding at the institution;
obtaining user decision input; and
receiving an intervention according to received user decision input based on displaying most crucial determinants, calculated aggregation and calculated interventions, for mitigating the crowding.

8. The institution having software and hardware for improving resource utilization, according to claim 7, further comprising computer-readable program code logic means and special purpose hardware adapted to perform the steps above.

9. The institution, according to claim 7, wherein improving resource utilization comprises improving utilization of one or more of doctors, waiting times, nurses, space, number of examination rooms, equipment and/or diagnostic services.

10. The institution according to claim 7, further comprising a hospital department.
